(19) United States
(12) Patent Application Publication
(10) Pub. No.: US 2019/0091687 A1
(43) Pub. Date: Mar. 28, 2019

(54) SAFE BLOOD PREGNANCY TEST AT HOME

(71) Applicant: Maha Abdullah A. ALHASNANI, Warwick, RI (US)

(72) Inventor: Maha Abdullah A. ALHASNANI, Warwick, RI (US)

(21) Appl. No.: 15/719,005

(22) Filed: Sep. 28, 2017

(51) Int. Cl. B01L 3/00; G01N 33/76

(52) U.S. Cl. CPC B01L 3/502753; G01N 33/76; B01L 3/50273; B01L 2300/0816; B01L 2300/0681; B01L 2200/10; B01L 2300/0627; B01L 2400/049; B01L 2400/0487; B01L 2400/0424; B01L 2400/0406; B01L 2200/0647; G01N 2800/36

(57) ABSTRACT

The invention is directed to a microfluidic chip, device, system, and method for conveniently detecting human chorionic gonadotropin in a blood sample without the need for drawing blood with a needle.

### GRANT OF NON-EXCLUSIVE RIGHT

This application was prepared with financial support from the Saudi Arabian Cultural Mission, and in consideration therefore the present inventor(s) has granted The Kingdom of Saudi Arabia a non-exclusive right to practice the present invention.

### BACKGROUND

### Field of the Disclosure

The invention is directed to a point-of-care microfluidic chip, device, system, and method for rapidly detecting human chorionic gonadotropin (“hCG”) in cell-free plasma obtained from small samples of human blood.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

hCG Testing

Tests for human chorionic gonadotropin (“hCG”) are employed for several reasons including to determine whether a woman is pregnant, detect a normal pregnancy, detect a ectopic pregnancy, detect and follow a molar pregnancy, determine risk of birth defects such as Down syndrome, or detect and follow cancers, especially germ cell cancers such as cancer of the ovaries or testicles or cancers which develop from germ cells such as eggs or sperm cells.

Conventional pregnancy tests detect hCG. An egg is normally fertilized by a sperm cell in a fallopian tube. Within 9 days the fertilized egg moves down the fallopian tube into the uterus. It then attaches (implants) to the wall of the uterus. After the fertilized egg implants, the growing placenta starts releasing hCG into the blood. Some hCG also gets passed in urine. HCG can be found in the blood before the first missed menstrual period. This can be as early as 6 days after the egg implants. hCG helps to keep a pregnancy going. It also affects the development of a baby (fetus). Levels of hCG go up fast in the first 14 to 16 weeks after the last menstrual period. They are the highest around the 14th week following the last period. They then go down gradually. The amount that hCG goes up early in pregnancy can give information about a pregnancy and the health of a baby. Soon after delivery, hCG can no longer be found in the blood. More hCG is released in a multiple pregnancy, such as twins or triplets, than in a single pregnancy. Less hCG is released if the fertilized egg implants in a place other than the uterus, such as in a fallopian tube. This is called an ectopic pregnancy.

hCG may also be measured as part of a screening test for birth defects. The level of hCG in the blood is often used as part of a screening for birth defects in a maternal serum triple or quadruple screening test. These tests are usually done between 15 and 20 weeks of pregnancy to check the levels of three or four substances in a pregnant woman's blood. The triple screen checks hCG, alpha-fetoprotein (AFP), and a type of estrogen (unconjugated estriol, or uE3). The quad screen checks these substances and the level of the hormone inhibin A. The levels of these substances-along with a woman's age and other factors-help the doctor estimate the chance that the baby may have certain problems or birth defects.

hCG is produced by certain tumors, especially those that come from an egg or sperm (germ cell tumors). hCG levels are often tested in a woman who may have tissue that is not normal growing in her uterus. The test also may be done to look for molar pregnancy or a cancer inside the uterus. Several hCG tests may be done after a miscarriage to be sure a molar pregnancy is not present. In a man, hCG levels may be measured to help see if he has cancer of the testicles.

A human chorionic gonadotropin (hCG) test is done to check for the hormone hCG in blood or urine. Quantitative hCG tests measure the level of hCG present at or above a particular threshold value. Non-quantitative tests just check to see if the hormone is present. Normal men and normal non-pregnant women have a very low hCG levels because hCG is made by the placenta during pregnancy and by cancer cells in abnormal conditions.

The hCG urine test is commercially available and is widely used to determine pregnancy. The urine test shows whether hCG is present (a yes-or-no result is indicated), but it does not quantify the amount of hCG, and can be insensitive and unreliable especially in the early stages of pregnancy.

hCG Levels. The ranges of hCG concentration may vary when measured using different assays, however, generally hCG levels range from less than 5 international units (“Ii”) per liter (5 IU/L) for men and non-pregnant women, from 5 to 50 IU/L for pregnant women a 1 week of gestation (about 3 weeks after the last menstrual period or LMP), 100 to 10,000 IU/L for pregnant women at 3 weeks gestation (about 5 weeks after the LMP), 1,080-30,000 IU/L for pregnant women at 4 weeks gestation (about 6 weeks after the LMP), 3,500 to 115,000 IU/L for pregnant women at 6-8 weeks gestation (about 8-10 weeks after the LMP), 12,000 to 270,000 IU/L for pregnant women at 12 weeks gestation (about 14 weeks after the LMP). With regard to the invention as disclosed herein, these ranges include all intermediate subranges and values, for example, each range includes subranges of about 10, 20, 30, 40, 50, 60, 70, 80, 90 and 100% of the described interval. Thus, the range 100 to 10,000 IU/L includes subranges of about 100, 1,000, 2,000, 3,000, 4,000, 5,000, 6,000, 7,000, 8,000, 9,000 and 10,000. A threshold detection value for a non-quantitative test may be set at any of these values intermediate values. Detection of a particular threshold value of hCG level or a quantitative hCG reading within the pertinent ranges above can indicate pregnancy, a particular probability of pregnancy, or the presence of an abnormal condition.

Conventional hCG tests. Conventional urine tests for hCG which can be performed by a patient at home are insensitive and do not always accurately detect amounts of hCG in early pregnancy as some women get false negative results. While blood tests for hCG can be more accurate, they can require the drawing of venous blood at a hospital or doctor's office and subsequent laboratory analysis.

Newer tests that potentially can be performed at home may not be easily accepted by persons afraid of needles or lancets needed for producing a blood sample. US20110253224A1 relates to a microfluidic system that can test for levels of hCG in blood. US20110172508A1 relates to a device with a microfluidic system that can test for levels of hCG in blood. US20140273187A1 relates to a point-of-care sensor system that includes portable readers and disposable cartridges for receiving and analyzing samples. US20120258472A1 relates to a microfluidic device and a system that detects hCG in blood. US20150044707A1 relates to an at-home blood pregnancy test kit that can detect hCG in blood. Clinical Lab Products newsletter at www.clpmag.com (Dec. 20, 2016) refers to rapid diagnostic tests for hCG and Abbott is developing the i-STAT® Total β-hCG test. However, home blood tests for hCG that are needle- and stab-free (lancet-free), safe, accurate, reliable, convenient, and acceptable to those afraid of needles or lancets are not available.

The present invention satisfies this demand by providing an easy, convenient, and safe microfluidic at-home test for hCG that does not require venipuncture or stabbing with a lancet. It provides accurate and valid results for women who don't get reliable results from urine tests, facilitates more frequent testing for subjects have conditions were close monitoring of hCG is desirable, is easy to use and requires very little expertise, reduces the cost and expense of medical care, does not require venous blood drawing and can be used at home even by people afraid of needles (trypanophobics) or lancets. The invention is fast, does not require time to produce serum from a blood sample and provides an immediate read out of the hCG assay result. A fast hCG test is highly desirable for an subject anxious about whether she is pregnant as well as for anxious or expectant parents and other intimate family members and friends.

### SUMMARY

A prominent embodiment of the invention is directed to a microfluidic chip and device that provides point-of-care or home testing for hCG in a subject's blood. A small amount (≤25 μl) of blood is obtained without venipuncture (needleless collection) by application of a delicate vacuum to a capillary bed close to the surface of the skin of the subject. Venipuncture or finger stab with a lancet is not required. This microfluidic chip is composed of a network of microchannels that links an inlet region where that receives the small amount of whole blood with a cross-flow filter that uses capillary forces to separate plasma from blood and/or a series of dead-end filters powered by micropumps (filtration stations, see FIG. 3) that remove large, medium and small blood cells and other solid components from whole blood to provide plasma. The plasma is then contacted with a detection region containing antibodies which produces a colorimetric or other detectable signal when bound by hCG in the plasma. The chip may be incorporated into a device comprising a signal display so that a user can determine whether a detectable signal has been emitted by the detection region. In some embodiments, the device will include a signal processor, clock, and/or information storage that provides a yes-or-no reading or that quantifies, graphs or otherwise processes a signal emitted by binding of hCG to antibodies in the detection region. For example, the device may compare and display using different colors the hCG reading and previous readings against a graph of a standard hCG hormone curve for a pregnant woman as shown by FIG. 2B, or notify a user when it is time for a new hCG reading or it may calculate and display the probability of pregnancy.

The information storage may be local or remote. The device may record or save medical data about the subject including the subject's name, patient ID or file number as well as the date and hCG test results. In a further embodiment, the device includes a connection, such as a wireless connection, that transmits the hCG test result information to a central source such as to a server in a hospital or doctor's office as depicted by FIG. 2B. The body of the device may be constructed from as few as six injection-molded parts. In other embodiments the device may be a cell phone, laptop, biometric or other electronic device designed and programmed to interface with the microfluidic chip and receive and display an hCG reading.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1. Illustrates several different cell types, including circulating tumor cells (“CTCs”), erythrocytes (red blood cells, “RBCs”) and various kinds of leukocytes (white blood cells) and describes their diameters. To produce blood plasma for analysis using the microfluidic chip of the invention, cells are removed from a blood sample, for example, by filtration through a series of filters that remove cells within different size ranges.

FIGS. 2A, 2B and 2C, respectively show top, bottom and side views of one embodiment of an integrated microfluidic chip and device according to the invention. One side of the device accommodates or contains microfluidic chip having a pathway into which blood is introduced (FIG. 2A). The obverse side (FIG. 2B) contains elements of a device that analyze, display, transmit, or store test result readout from the microfluidic analysis. FIG. 2C depicts a side-view of the device. In this embodiment, the microfluidic chip forms only a part of the surface of the device. In a preferred embodiment, the device is designed so that the microfluidic chip component is replaceable thus facilitating repeat testing and use of the device to receive, display or record multiple hCG readings. The device may also accept microfluidic chips that detect other analytes and be configured to be worn or hand-held.

FIG. 3 depicts a microfluidic platform with an inlet that applies a gentle vacuum and a fluid pathway with three filtration stations through which blood plasma passes before reaching the detection area.

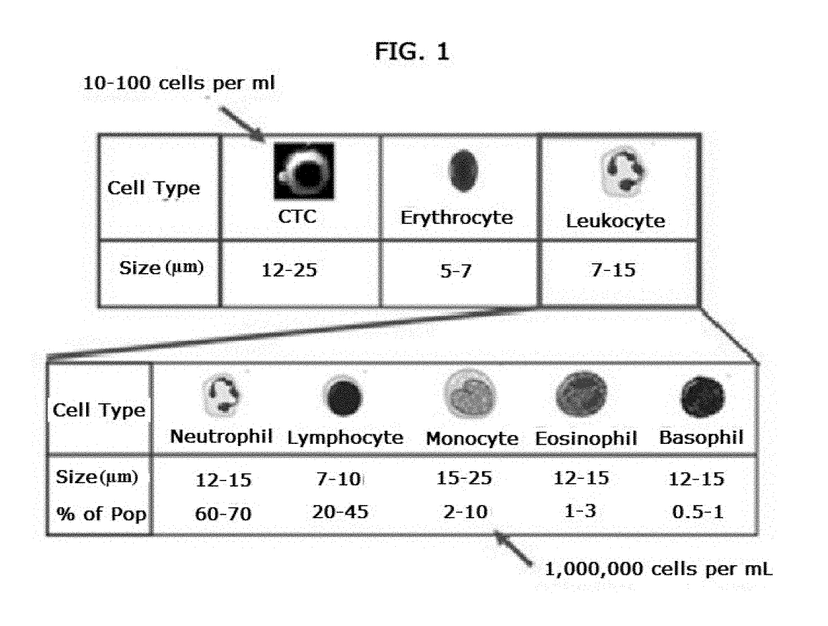

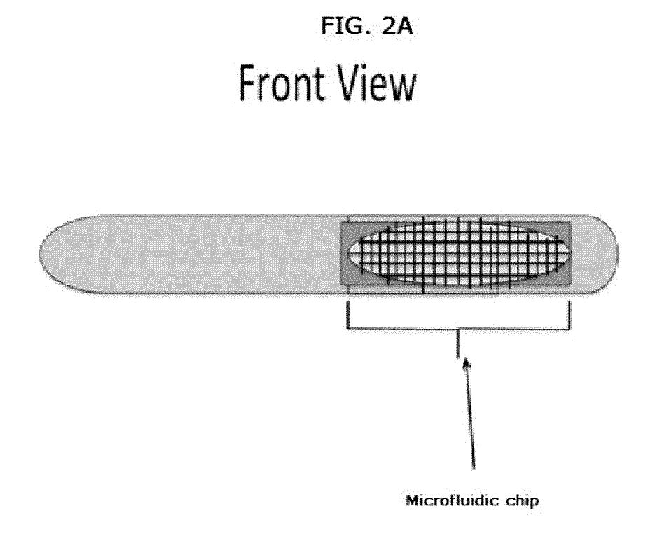

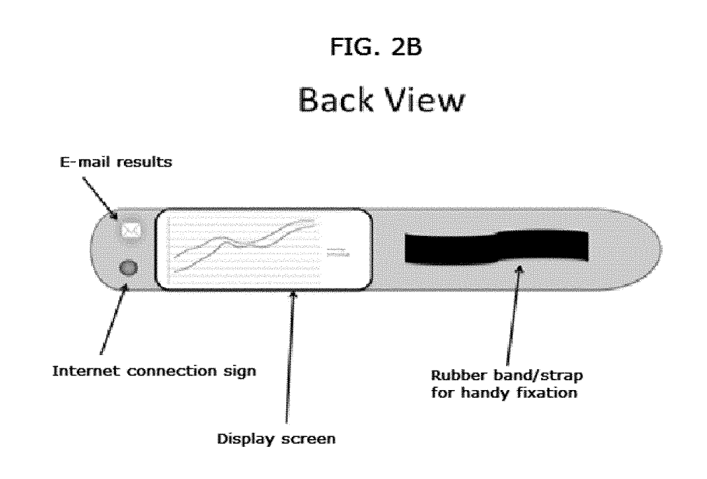

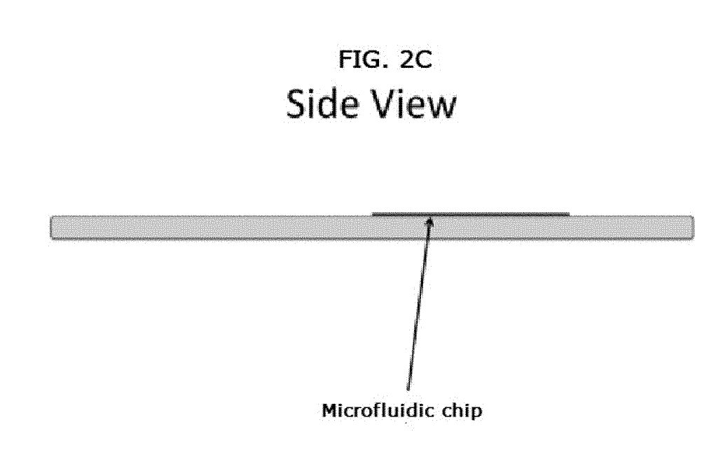

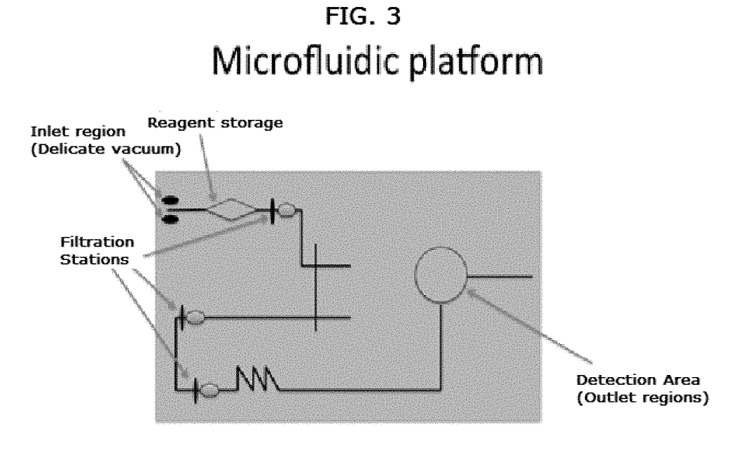

### DETAILED DESCRIPTION OF THE EMBODIMENTS

Referring now to the drawings, wherein like reference numerals designate identical or corresponding parts throughout the several views.

A prominent embodiment of the invention pertains to a microfluidic chip, device, and method for detection of human chorionic gonadotropin or hCG, a hormone that is elevated during pregnancy and certain other abnormal conditions. This device may be used to measure hCG levels in blood at home or at a point of care. The term “point-of-care” as used herein is defined to mean a location on or near a site of patient care where medical testing and/or treatment can be performed. For example, locations for point-of-care may include but is not limited to, hospitals, patient homes, the home of a friend or relative, a physician's office, or a site of an emergency.

The hCG detection method does not require the collection of venous blood by venipuncture and is suitable for use by people having a fear of needles or lancets. In a preferred embodiment, the device collects only a very small amount of blood on the order of 25 microliters or less from a capillary bed under a mild vacuum. Unlike conventional blood tests for hCG, it is not necessary to draw venous blood, coagulate it, and separate blood serum for hCG testing. The device of the invention separates blood plasma from the cellular components of blood and detects hCG in blood plasma. These features are advantageous for a point-of-care or at-home hCG blood test which offers convenience as well as better accuracy than conventional at-home urine tests for hCG.

The term “molecular diagnostics” or “diagnostic testing” as used herein is defined to mean any medical or biological testing that may be performed on a biological sample for analytical or diagnostic purposes. For example, the biological sample may include, but is not limited to, a blood, plasma or serum sample; the analyte is hCG or the beta subunit of hCG, though the chip and device may be adapted to detect other analytes by selection of an antibody or other ligand that binds to the other analyte.

Blood Collection. Conventional medical blood tests for hCG obtain blood by venipuncture. A patient's arm is wrapped with a tight elastic band, the site of venipunction is wiped with alcohol or another topical germicide, a needle is inserted into the vein, and venous blood withdrawn through the needle. Venipuncture techniques are incorporated by reference to: https://_en.wikipedia.org/wiki/Venipuncture (last accessed Jul. 10, 2017). Drawbacks of conventional blood collection involve the time and cost to have blood professionally collected at hospital, doctor's office or other health facility, resistance or non-compliance by patients afraid of needles (trypanophobia) or skin stabs, fainting, lightheadedness, bruising around the anatomical site of collection, hematoma, phlebitis or swelling of a vein after blood collection, and excessive bleeding by patients taking blood thinning medicines or agents such as aspirin or warfarin. Other drawbacks of conventional medical blood tests measuring hCG include the delay and cost of processing a sample, perceived loss of privacy, and the requirement to produce and submit a medical claim.

Some blood tests are conducted on small quantities of venous blood obtained by finger stick or finger prick, or for some neonates by heel prick. A blood lancet is a small medical implement used for capillary blood sampling. Lancets are used to make punctures, such as a finger stick, to obtain small blood specimens. Blood lancets are generally disposable. A site free of surface arterial flow where the blood is to be collected is sterilized with a topical germicide and the skin pierced with a sterile lancet. After a droplet has formed, venous blood may be captured by a test strip, capillary tube, or other device often through wicking or capillary action. Blood from a finger stick may be applied to a test strip measuring blood glucose levels and read with a meter or used to obtain DNA for genetic testing. While less invasive than venipuncture, finger stabs with a lancet are messy and not acceptable to some subjects.

Needleless Blood Collection. In contrast to conventional venipuncture, the invention collects blood from a capillary bed close to the surface of a skin under a gentle vacuum. Such needle-less blood collection techniques are described by HemoLink™ (http://_www.tassoinc.com/vision/), US 2016/0174888 A1, or by U.S. 2015/0342509, “Needle-free Blood Draw” both of which are incorporated by reference, however, they fail to provide the features necessary for a blood-based home pregnancy test including integrated collection, plasma separation, filtration, pumping, and hCG detection features.

The element for needleless blood collection may be part of the microfluidic chip, part of a device attached to the microfluidic chip, or a stand-alone component. Preferably, this blood collection element is integrated into the microfluidic chip or device. The blood collection element in addition to supporting a vacuum, may contain abrasive or microincisive elements, analogous in size, shape and/or function to mouth parts of a leach, to facilitate removal of blood by the vacuum. This element may also be coated with or applied along with a topical anesthetic (such as benzocaine, butamben, dibucaine, lidocaine, oxybuprocaine, pramoxine, proparacaine, proxymetacaine, and tetracaine), antibiotic or disinfectant, antihistamine, or anticoagulant (e.g., heparin, hirudin, coumarins, Xa inhibitors, thrombin inhibitors, EDTA, citrate, oxalate, or Vitamin E) to facilitate the safe, painless, and efficient removal of blood using a vacuum. Other useful agents include Citrate Dextrose Solution or “Anticoagulant Citrate Dextrose Solution and Warfarin. Puncture of a vein or stabbing with a lancet is not required and thus this blood collection method will be acceptable to many subjects or patients. In some embodiments, blood may be contacted or admixed with the above agents during or after its collection, or such agents may be applied to surfaces involved in blood collection. Such agents or their dosages are generally selected that would not place a pregnant woman or her fetus at risk.

Chemical additives help maintain the plasma in liquid form, facilitate its movement through the device to the detecting region, and generally improve capillary fluidity. Antihistamines or anticoagulants also facilitate the efficient removal of blood using a vacuum to assist in plasma separation and hCG detection.

Blood, plasma, and serum. The term “blood components” describes components that may be found in a sample of blood. For example, the blood components may include, but are not limited to, white blood cells (WBCs), red blood cells (RBCs), platelets, plasma, and bacteria.

Plasma is usually produced by collecting whole blood in tubes that are treated with an anticoagulant so that the blood does not clot. The cells are then removed by centrifugation and the supernatant, designated plasma, carefully removed from the resulting cell pellet using a Pasteur pipette. While plasma does include clotting factors it can also contain measurable amount of hCG. The invention collects blood and produces plasma using microfluidic processes. In some embodiments of the invention a microfluidic chip will contain an anticoagulant to facilitate separation of plasma from blood or detection of hCG.

Serum is the liquid fraction of whole blood that is collected after the blood is allowed to clot. Generally clotting takes at least 15-30 mins. The clot may be removed by centrifugation and the resulting supernatant, designated serum, removed using a Pasteur pipette. The invention conveniently uses plasma instead of serum, however, in some embodiments a serum sample may be used, e.g., when the device is used to test a stored serum sample.

Conveniently for home testing the biological sample applied to the microfluidic chip of the invention is whole blood. However, other fluids to be assayed for human chorionic gonadotropin may also be applied, for example, in a doctor's office where serum or other non-whole blood samples may be available. These include but are not limited to plasma, serum, cerebrospinal fluid, lymph, follicular fluid, amniotic fluid, testicular fluids (e.g., from hydroceles), fluids associated with cancer or tumors, saliva, and urine.

Micropumps. The effects of physical laws change within the microfluidic domain. As an example, volumetric forces, such as weight or inertia, often become negligible, whereas surface forces can dominate fluid behavior. Most micropumps rely on principles of micro-actuation which can be scaled up only to a certain size. Micropumps are grouped into mechanical and non-mechanical devices. Mechanical systems contain moving parts that are usually actuation and valve membranes or flaps. A driving force for a mechanical system can be generated using piezoelectric, electrostatic, thermo-pneumatic, pneumatic or magnetic effects.

Non-mechanical pumps function with electro-hydrodynamic, electro-osmotic, electrochemical or ultrasonic flow generation. Non-mechanical pumps that are chemically powered may have nanomotors fixed to surfaces to facilitate a chemical reaction powering the pump. A wide variety of pumping systems exist ranging from enzyme-based pumps to those using an organic photocatalyst or those using a metal catalyst. These pumps generate flow through a number of different mechanisms including self-diffusiophoresis, electrophoresis, bubble propulsion and the generation of density gradients.

In microfluidics, capillary pumping plays an important role because the pumping action does not require external actuation. Glass capillaries and porous media, including nitrocellulose paper and synthetic paper, can be integrated into microfluidic chips. Capillary pumping is widely used in lateral flow testing and can be used in methods that separate a liquid from solid components in a biological sample. Capillary pumps, with a constant pumping flow rate independent of the liquid viscosity, have been developed, which have a significant advantage over the traditional capillary pump because their performance does not depend on the sample viscosity.

Miniaturized devices, obtained with micromachining techniques, into which a blood sample is introduced may be used to detect hCG. Some of these microfluidic devices, generically known as “Lab-On-Chip” or “Lab-On-a-chip” (LOC) or “Micro Total Analysis Systems” (μTAS), are known as “microfluidic devices” because they integrate one or more miniaturized hydraulic components, such as paths, passages, valves, filters, and obstacles, which have at least one characteristic such as length, width, height, thickness, section of passage, etc., of less than 1 mm. For example, an intake for a biological sample, pathway between an intake and a filter, or pathway from a filter to a detecting region, or a detecting region may be dimensioned to have a height, width, or length of 50, 100, 200, 250, 300, 350, 400, 500, 600, 700, 800, 900 or ≤1,000 μm or any intermediate value or subrange within this range. In other embodiments a microfluidic chip may comprise smaller or larger dimensions, such as 1, 2, 3, 4, 5 or more millimeters, so long as the fluid that is received, conducted, processed or detected can move through the chip, for example, by capillary action or by microfluidic pumping.

In some embodiments, the microfluidic channels may comprise smaller or larger dimensions, such as 1, 2, 3, 4, 5 or more millimeters, so long as the fluid that is received, conducted, processed or detected can move through the chip by capillary action or microfluidic pumping. The aspect of height-width ratio is an important parameter for successfully injection molding micro-channels. Tall and thin channels are usually preferred for fluidic functionality, for example, channels having a height to width ratio of >1:1, 1.5:1, 2:1, 3:1, or 4:1. When one wants to use gravitational force in the direction of fluid flow, then microfluidic channels can be designed with shorter and wider channels as microchannel design influences the effects of gravity and/or capillary forces.

Designing a channel that utilizes a combination of capillary and gravity forces can be accomplished by changing the geometry of the channel, properties of the fluids, and device materials. The Bond number can be used to design these channels from the Equation: Bo=ΔρgL 2/σ where Δρ is the difference in fluidic density between the fluid flowing in the channel and the fluid surrounding it, g is the gravitational constant, L is the characteristic length of the channel, typically its width, and σ is the surface tension of the fluid. For Bond numbers lower than 0.1, capillary forces serve as the primary driving forces, and gravity is of lesser influence. At Bond numbers above 10, gravity becomes the primary driving force. For Bond numbers between 0.1 and 10, both capillary and gravitational forces have a definitive effect that can compete, amplify, or alter one another. Finally, capillary and gravitational forces can be used in conjunction in the design of channels, so as to enhance and otherwise direct the flow of a collected fluid.

Rendering that information, a channel can be narrow (less than 1 mm) of high capillary force (a low Bond number) or wide of high gravity force where the Bond number is higher. Subsequently, to drive the device by capillary forces, fluid may be readily drawn into the narrow channel due to the high capillary force that dominates gravity, which is ultimately based on channel geometry and measurements. The ability to utilize capillary and gravitational forces together to create efficient channels can result in devices that are simpler, less expensive, and easier to manufacture and more robust in their operation because they have higher working tolerances, therefore not requiring as much precision in the channels, which results in unit cost reduction.

In preferred embodiments of the device of the invention, fluid flow is driven by capillary force solely and channels can be designed with dimensions less than 1 mm permitting free positioning of the device during testing without the need for gravity driven flow, such as that needed to move fluid in a main channel of devices such as the Hemolink system.

Filtering. The term “microfluidic filter” describes a filter comprising a porous or perforated surface used to process, purify, or otherwise manipulate a fluid constrained to a small (e.g., less than 5 mm) diameter. The term “pore” as used herein is defined to mean any opening through which matter, commensurate in size with or smaller than the opening, can pass.

Dead-end filtration_refers to filtration in which a fluid flow pass through a filtering surface that retains solid particles or cells in the fluid flow. An example would be passage of a flow of blood cells and plasma perpendicularly through a filter that retains blood cells but permits plasma to flow through the filter. There is no restriction of the blood-filtering material used in the invention. A single-type of dead-end filter may be used that progressively entangles large blood cells, then smaller blood cells, and then smaller blood components. Alternatively a series of filters having different pore sizes may be used. The filters may entangle blood cells within them or trap blood cells at their surfaces. In some embodiments a glass fiber filter, a microporous membrane or combinations of both are used.

A glass fiber filter can have a density of about 0.02 to 0.5 g/cm³, preferably about 0.03 to 0.2 g/g/cm³, more preferably about 0.05 to 0.13 g/g/cm³, and a retainable particle size of about 0.6 to 9 μm, preferably 1 to 5 μm. The surface of glass fiber filter may be treated with hydrophilic polymer to facilitate faster and smoother action. A lectin, anticoagulant, or other reactive reagent or modifier may be incorporated into the glass fiber or the glass fiber may be treated therewith. Two or more sheets of a glass fiber filter may be stacked.

Microporous membranes with hydrophilic surfaces may also be employed for separating blood cells from whole blood to obtain plasma. A suitable pore size of the microporous membrane is smaller than the retainable particle size of glass fiber filter but is 0.2 μm or more, preferably about 0.3 to 5 μm, more preferably about 0.5 to 3 μm. A higher void content of the microporous membrane is preferable, and a suitable void content is about 40 to 95%, preferably about 50 to 95%, more preferably about 70 to 95%. The microporous membranes include but are not limited to those comprising a polysulfone membrane, fluorine-containing polymer membrane, cellulose acetate membrane, and nitrocellulose membrane.

Human red blood cells are disks of diameter 6-8 microns and thickness 2 microns and have an occurrence of approximately 5 million cells per μL of blood. Human leukocytes, comprising mostly neutrophils (approximately 60%, 10-12 micron diameter) and lymphocytes (about 30%, 7-8 microns diameter) have a total occurrence of about 4,000-11,000 cells per μL of blood. Human platelets (thrombocytes) are 2-3 micron diameter and have an occurrence of 150,000-400,000 cells per μL of blood. A red blood cell can squeeze through openings smaller than 6 microns by deformation and may be passed or excluded by selecting a size necessary to affect their physical separation from other blood components. For example, a set of openings of size 5 microns may block passage of most leukocytes, but may allow deformable red blood cells and platelets to pass. Analogously, a filters having a pore size of 2-3 micron can allow platelets to pass but block passage of red blood cells. Smaller pore size filters can be used to block passage of platelets and let plasma pass.

In one embodiment, the invention withdraws whole blood and employs dead-end microfilters to sequentially remove blood cells having different sizes. For example, microfilters with different pore sizes are used to remove red blood cells having a disk diameters ranging from approximately 6.2-8.2 μm and a thickness at the thickest point of 2-2.5 μm; white blood cells including neutrophils and eosinophils ranging from about 10-12 μm; basophil and large lymphocytes ranging from about 12-15 μm; small lymphocytes ranging from about 7-8 μm; monocytes ranging from about 15-30 μm and platelets ranging from about 2-3 μm in greatest diameter. Microfilters may be selected to retain one or more of these kinds of cells and permit smaller cells and plasma to pass through. Preferably, larger cells are removed first. As plasma contains coagulants, an anticoagulant such as heparin, may be contained in a microfilter or in other portions of the microfluidic chip to inhibit coagulation of blood cells while they are being sequentially removed from whole blood. In some embodiments the smallest or ultimate filter will have pore sizes of about 0.02 μm.

This filtration system can process whole blood and produce cell-free plasma from small quantities of blood, such as 50, 25, 20, 15, 20, 10 or 5 μLs of whole blood. In preferred embodiments it will produce 1-5 μl of plasma. It is understood that the cell and pore size ranges above include all intermediate subranges and values, such as about 1, 2, 3, 4, 5, 6, 7, 8, 9, 10, 12, 15, 20, 25, and 30 μm in maximum or average diameter.

The pore sizes of sequential microfilters may be selected to improve the amount of plasma produced and to minimize clogging of the filters. Filtration may be powered by capillary forces and/or by micropumps arranged before or after filters. However, in some embodiments, the device will provide positive or negative pressure to the microchip to allow blood and plasma to flow through the chip.

Cross-flow filters. The term “cross-flow filtration” refers to filtration in which a fluid flow direction is parallel to a filtering surface. An example would be flow of blood through a tubular conduit where the filtering surface forms the walls of the tube and is permeable to plasma but not blood cells. In this example, plasma would be removed in a direction away from the axis of the tube, while blood cells could continue movement through the tubular conduit. In some embodiments, the device of the invention will incorporate a cross-flow filter or separate blood cells from plasma by diaelectrophoresis as described, for example, by Chen, et al., Lab. Chip, 2014, 14 (2014), Chen, et al., Microelectronic Engineering 128: 36-41 (2014), by Szydzik, Crispin, et al., Biomicrofluidics 9.6: 064120 (2015), or by Shim, et al., 14th International Conference on Miniaturized Systems for Chemistry and Life Sciences 3-7 Oct. 2010, pp. 145-147, which are incorporated by reference. The term “dielectric separation” refers to a method of separating different types of substances or cells based on a difference in charge that may be either inherent and/or induced. A dielectric separation can include, but is not limited to, separation of blood cells or other solid components of blood from plasma.

Dielectrophoretic methods for cell and particle separation are based on the polarization of suspended particles or cells. A microfluidic network using this method can be a capillary-driven, portable, and low voltage cDEP plasma separation chip that can have asymmetric capacitive-coupled electrodes to induce inhomogeneous electrostatic forces in the microchannel and increase dipole-dipole interactions between the red blood cells. A cDEP plasma separation chip can separate plasma from undiluted human whole blood samples without any external driven force. The intensity of capillary forces affect plasma separation time and the velocity of the sample. In some embodiments blood cells may be separated from whole blood by dielectrophoretic methods as well as filtration, for example, red blood cells may be removed using dielectrophoresis and other blood components by filtration.

Antibodies. Antibodies to hCG and hCG beta subunit suitable for ELSA and other diagnostic procedures are well known and publically available, for example, via Thermofisher at https://_www.thermofisher.com/antibody/primary/target/hCG (last accessed Jul. 21, 2017) which is incorporated by reference.

Detection. ELISA and other methods for detecting the binding of hCG or its beta subunit to anti-hCG or anti-hCG beta subunit antibodies are known. The microfluidic chip or device can use enzyme-linked immunosorbent assay (ELISA) that utilizes monoclonal hCG antibodies that bind to hCG in the sample leading to its interaction with enzyme-linked antibodies and dye substrates that produce a color reaction when the antibodies are activated or it can use other immunologic chemical reactions. Most chemical tests for pregnancy look for the presence of the beta subunit of hCG in the blood. Intact hCG molecule consists of two subunits; the alpha and beta subunits, that are similar to luteinizing hormone, follicle stimulating hormone, and thyroid stimulating hormone. As the alpha subunits of these hormones are identical and their beta chains are unique, testing for the presence of hCG using antibodies to the beta subunit enables the presence of hCG to be distinguished accurately. In one embodiment, binding or complex formation between hCG in a plasma sample and antibodies to hCG or its beta subunit produces a colorimetric signal or radioactive signal via an indicator of complex formation. In some embodiments, upon binding of hCG to antibodies in the detecting region and complex formation, a second tagged antibody to hCG beta subunit is introduced into the detecting region from a reservoir, binds to hCG in the complex, and provides a detectable signal of complex formation. A sensor detects the degree of color change or change in radioactivity above a particular threshold and displays a green (positive for hCG) or yellow, orange or red signal for an hCG value below the threshold.

An immunologic detection method for binding of hCG to antibodies to hCG or hCG beta subunit include any immunologic chemical reaction that can measure the formation of antibody-antigen complexes and detect them via an indicator reaction. These methods include but are not limited to radioimmunoassay, enzyme (EIA) immunoassay, enzyme-linked immunosorbent assay (ELISA) and chemiluminescent immunoassays. Direct, indirect, or sandwich assays may be used.

Radioimmunoassay technology integrates radiolabels like I-125, Zn-65 (or any safe detectable radiolabel) to a specific antigen-antibody binding, then, when hCG antigen is coming from the sample, it will compete with the specific manufactured antigen leading to its detachment thereby detection of its concentration. However, this technique is expensive, requires sophisticated equipment, special radiation precautions and medical personnel. While its use in a microfluidic chip is advantageous because it is so accurate and sensitive, a home test should inform the user of certain safety and disposal precautions. Enzyme (EIA) immunoassays were developed as an alternative to radioimmunoassay (RIA). These methods use an enzyme to label either the antibody or antigen. Enzyme-linked immunosorbent assays (ELISA) and chemiluminescent immunoassays utilize a chemiluminescent label. Chemiluminescent molecules produce light when they are excited by chemical energy. A light detector measures these emissions.

Device Features. The device of the invention may incorporate functional design features that make its use convenient, comfortable, practical and to facilitate interaction between the device and the user. Such users include women desiring to determine conception and pregnancy as soon as possible or a patient having an abnormal condition requiring periodic monitoring of hCG level.

In some embodiments the device will be designed to comfortably fit against a mucosal lining having a capillary bed close to the surface so that a blood sample can be obtained from capillaries in the mucosal lining and then processed and assayed by the device. In these embodiments, the device may have an external cover that is smooth with no sharp edges of faces, no cracks, holes or pits which might retain biological materials, and have a handle, tie or other feature to permit removal after insertion. The shape of the device may be cylindrical, smooth, or have tapered ends and be otherwise conformed to comfortably fit into an anatomical location having a mucous membrane such as the nose, mouth or vagina. It may have a handle, extension, waist, or other positioning element to permit it to be maneuvered into or out of an anatomical opening and held against a mucosal surface. It may also be made of a material that is biocompatible, non-irritating, hypoallergenic, and sanitizable. Only the part of the device that obtains the blood sample need be inserted into the body.

The device may be designed to cooperatively attach to a disposable microfluidic chip and to hold additional microfluidic chips for later use. Thus it may include a detachable case or cover to hold or protect the device, stock of microfluidic chips, and instructions for performing the hCG assay and using the other features of the device.

The device may contain a power source, such as a lithium battery, a pump or other element that provides a vacuum or positive pressure to a housed microfluidic chip. It may include a cleanable or clearable pathway or a removable waste reservoir to remove of hold blood cells and other biological wastes produced by the microfluidic chip. In embodiments where portions of the device that collect or transfer blood into the microchip, the device may be designed to be sanitized between readings or may be designed to have a separate, replaceable blood collection cassette independent of the device and microfluidic chip, such as a replaceable blood collection pod that is seated on the device, receives blood and transfers it to the microfluidic chip.

The device may be designed to be securely held in the hand or sized to be comfortably worn on the wrist, arm, or elsewhere on the body. For example, it may comprise a band, loop, or bracelet that secures it to the wrist or arm. Its features may be smoothed and contoured so that it presents no sharp edges or faces or it may be designed and colored to be inconspicuous when worn, for example, it may be flesh colored, match the color of clothing, or designed to look like a watch or other jewelry. It may be sized or packaged to fit within a woman's purse or man's wallet or boxed and encased in a cosmetic wrapping as a gift.

The description, embodiments and specific examples describe non-limiting embodiments of the technology and intended for purposes of illustration only, but are not intended to limit the scope of the technology. The recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

The invention is directed to a microfluidic chip containing an inlet for receiving blood to be assayed for human chorionic gonadotropin, a cross-flow filtration system for removing blood cells from plasma; and a detecting region comprising antibodies that bind to a beta unit of human chorionic gonadotropin hormone. Preferably, blood is collected using a gentle vacuum although blood from other sources may also be analyzed using the microfluidic chip. In this chip the inlet and cross-flow filtration system may be connected so that blood flows from the inlet into the filtration system and the filtration system and detecting region are connected so that plasma flows from the filtration system to the detecting region. In some embodiments, the chip contains or is coated or treated with materials that prevent coagulation of fibrinogen and other materials found in blood or plasma. This helps maintain the analyte, generally blood plasma, in a liquid form and facilitates its movement through the device to the detection region. The microfluidic element of the invention may be used by itself or in combination with a detection device such as a meter or an optical or audio detector that produce detectable signals when human chorionic gonadotropin binds to antibodies in the detection region of the microfluidic chip.

In another embodiment of the invention, the device contains (a) an inlet for receiving blood; (b) a microfluidic chip comprising a cross-flow filtration system for separating blood cells from plasma; (c) a micropump for effecting blood flow through the microfluidic chip; (d) a detecting region comprising antibodies that bind to a beta unit of human chorionic gonadotropin hormone; (e) an optical detector for detecting a change in color when the antibodies bind to the beta unit of human chorionic gonadotropin hormone (hCG); (f) an outlet for disposing the separated blood cells; (g) a wrist loop disposed on the exterior of the device and arranged parallel to the longitudinal axis of the device; (h) a retractable cover for the microfluidic chip; and (i) a contoured edge/face to make holding or wearing the device comfortable. Other embodiments include:

In one embodiment, the invention is directed to a device comprising a microfluidic chip where the device comprises a chamber that when applied to a dermal capillary bed can be placed under a vacuum sufficient to remove whole blood from the capillary bed, an inlet that receives non-venous blood from the chamber, a filtration system for separating blood cells from plasma, wherein the filtration system comprises (i) a series of filters that remove blood cells, platelets or other solid components of blood ranging in diameter from 12 to 30 μm, 5 to <12 μm, and 0.1 to <5 μm or any value within these ranges (in some embodiments, blood cell diameter ranges may overlap), or (ii) a cross-flow filtration system that separates plasma from whole blood; a detecting region comprising antibodies that bind to a beta unit of human chorionic gonadotropin hormone; and wherein the inlet and filtration system are connected so that blood flows from the inlet into the filtration system; and wherein the filtration system and detecting region are connected so that plasma flows from the filtration system to the detecting region.

In some preferred embodiments, the microfluidic chip will house the chamber, inlet, filtration system, detecting region and the microfluidic pathways connecting them. However, in some embodiments, one or more of these elements may be housed outside of the microfluidic chip, for example, the chamber for collection of blood using a vacuum or the detecting region may be located outside of the microfluidic chip which receives blood and processes into plasma. Thus, the chamber for collecting blood may be connected to a vacuum source outside of the microfluidic chip or plasma produced by the microfluidic chip may be fed to a detecting region outside of the chip.

In some embodiments the device or microchip comprises a series of micropumps that can drive blood or plasma when present in the chip through the filtration system and to the detecting region. In other embodiments the comprises one or more structures that draw blood or plasma when present in the chip through the filtration system and to the detecting region, for example, by inducing or providing capillary forces. Also the device may be connected to an external element that drives movement of blood or plasma when present in the chip through the filtration system by positive pressure applied upstream from the filtration system or by negative pressure provided downstream of the detecting region.

In some embodiments, the device comprises (i) a series of filters that remove blood cells, platelets or other solid components of blood ranging in diameter from 12 to 30 μm, 5 to 11 μm, and 0.1 to 5 μm. In other embodiments a solitary filter, or a series of two, three of more filters, may be arranged to progressively separate larger, then smaller blood cells from plasma.

In other embodiments, the comprises (ii) a cross-flow filtration system that separates plasma from whole blood. This cross-flow filtration system may employ an electric field to charge and remove blood cells from plasma and removal of plasma from a blood sample may be powered by capillary pumps or structures that induce capillary forces. For example, the device may comprise (ii) a cross-flow filtration system that separates plasma from whole blood and diaelectrophoretic electrodes that apply a charge to blood cells. The device may be configured so that the flow of blood to the filtration system or the flow of plasma from the filtration system to the detecting region is driven by capillary forces.

In most embodiments, the device will contain either or both of a cross-flow or dead-end filter as both types of filtration are suitable for use in the microfluidic chip and serve the ultimate goal of removing and retaining solid blood components from the plasma.

A cross-flow filter utilizes dielectric separation of different types of cells based on a difference in charge that may be either inherent and/or induced. Dielectrophoretic methods are based on the polarization of suspended particles or cells. Filtration can be conducted in portable, capillary-driven, and low voltage cDEP plasma separation microfluidic chip that has electrodes to induce inhomogeneous electrostatic forces in the micro-channel, increase dipole-dipole interactions between the red blood cells and more importantly, it separates plasma without external driven force.

Dead-end filtration refers to filtration in which a fluid flow pass through a filtering surface that retains cells in the fluid flow but permits plasma to flow through the filter. A series of filters having different pore sizes may be used. In some devices a glass fiber filter that retains a specific particle size and may treated with hydrophilic polymer to facilitate faster and smoother action. Also, a lectin anticoagulant, or other reactive modifier may be incorporated into the glass fiber, a micro-porous membrane that has hydrophilic surfaces and suitable pore size smaller than the retainable particle size of glass fiber filter, or combinations of both are used.

In some embodiments, red blood cells may be removed by dielectrophoretic methods and other blood components by filtration. Filtration may be powered by capillary forces and/or by micro-pumps arranged before or after filters. However, some devices may provide positive or negative pressure to the microchip to allow blood and plasma to flow through the chip. Cross-flow filtration system applied in some devices, plasma is removed from a blood sample by capillary pumps or structures that is induced by capillary forces.

The device may further comprise one or more outlets or reservoirs for removing blood cells, platelets, or other solid components of blood. These outlets or reservoirs may be designed so that they can be cleaned and sanitized between uses or may form part of a single use microfluidic chip or device.

Preferably, the detecting region of the microfluidic chip or the device emits a detectable signal when bound by the beta unit of human chorionic gonadotropin. The detecting region may comprise an indicator that exhibits a color when the detecting region is bound by the beta unit of human chorionic gonadotropin. In some embodiments, the inlet, filtration system, or detecting region comprises at least one anti-coagulant.

In some embodiments, the device is configured for external use, in others the device may be configured for insertion into the body so that a blood sample may be obtained from a capillary bed near the surface of a mucous membrane. In other embodiments the device configured to fit into a signal processing, display, data storage, or transmission device.

Another embodiment of the invention is a system comprising the device described herein and a vacuum source. The vacuum source may be integrated into the device or be a separate part of a system that includes the device. This system may further comprise an optical, radiation or other detector of a signal produced by binding of hCG in plasma to the antibodies in the detecting region; or comprise an optical detector that is operatively connected to a screen that visually displays a result or that comprises an audio element such as a speaker that speaks or otherwise produces an auditory signal of a result, wherein said result is produced by binding of hCG in plasma to the antibodies in the detecting region. This system may further comprise a signal processing, display, data storage, or transmission element, or be designed in the form of a bracelet, watch, arm band, mobile phone, computer, or other wearable or hand-held item.

A device may be configured to transmit or display a standard hormonal curve for a pregnant women in one color (e.g., blue), the hormonal curve for the subject in another color (e.g., red). The device may have or be attached to a memory element so that the results, test date, medical data, and other relevant information is saved. Such data may be manipulated, transmitted, or displayed using a touch screen on the device or on another device electronically connected (for example, via blue tooth type connection or Wi-Fi) such as a laptop, smart watch or cellphone. These devices may also contain electronic elements for transmission of results to a point of care center (UC) using a subject's fingerprint, retinal print, or other personal security data which is already verified in the UC System.

On the subject of fingerprinting (or other personal security data), the device can be integrated with an application installed at a central source such as to a server in a hospital or doctor's office to verify patient's identity via fingerprinting using the touch screen, to receive hCG test result information (including patients having an abnormal condition), and to pool up patient's data through a wireless connection. Data may also be connected to a mobile through Wi-Fi for patient to send their own information and be able to monitor themselves regarding time of test, results, ovulation days (e.g., detecting luteinizing hormone in conjunction with hCG detection), estimated conception day and due day in case of pregnancy. Hence, the device may have a fingerprint touch screen feature or scanning feature for other personal security data that insures patient identity and confidentiality as it is already have been verified at point of care systems and facilitates patient's personal and medical data recall.

Other ergonomic features may appear in other embodiments of the device of the invention. These include an attachment port to a disposable microfluidic chip and a slot, container or other structure to hold a stock of unused microfluidic chips for later use. Thus it may include a detachable case or cover to hold or protect the device. A replaceable microfluidic chip facilitates repeat testing and use of the device to receive, display or record multiple hCG readings. In some embodiments the device may include a cleanable pathway or a removable waste reservoir to remove of hold blood cells and other biological wastes produced by the microfluidic chip. Indeed, portions of the device that collect or transfer blood into the microchip may be designed to be sanitized between readings (e.g., with a chemical additive, disinfectant or sanitizer that goes through the chip after each run, either via a button or automatically) or may be designed to have a separate, replaceable blood collection cassette independent of the device and microfluidic chip.

In some embodiments, the device will be suitable for hearing a baby's heartbeat and may contain an audio sensor, such as a microphone, and audio output, such as a speaker. It may also contain data processing elements that record or analyze fetal heartbeat or the heart beat and blood pressure or other biometric information of a mother.

Another embodiment of the invention is a method for detecting human chorionic gonadotropin hormone in blood comprising contacting a blood sample with the device described herein and detecting a signal generated when human chorionic gonadotropin binds to the detection region of said device.

In other embodiments, a method or device according to the invention will use a complex network of microchannels linking the inlet and outlet regions of the device to receive a blood sample, extract plasma from the blood sample, and transport and test the plasma for hCG.

In most of these embodiments, the complex microfluidic network will provide s a blood collection device that contains blood collection sites, micro-needles, vacuums or pumps to collect the blood, it exploits a series of dead-end filtration systems powered by micropumps as well and/or a series of cross-flow filtration systems that use capillary forces, and in addition to hCG or other pregnancy hormone detectors provides colorimetric or electronic display or transmission of the results of pregnancy hormone detection. For example, it can display or transmit information describing a standard hormone curve for pregnant women in one color (e.g., in blue), and a tested hormone curve for the tested subject in a different color (e.g., red).

In other embodiments, the device and method according to the invention will incorporate or employ one or a combination of more than one distinguishing features including utilizing a mild or gentle vacuum at each blood collection site to collect a small (≤25 μl) blood sample and deliver it from a capillary bed into an inlet region or port of the device; utilizing abrasive or micro-incisive elements analogous in size, resembling leech teeth, to facilitate removal of blood by puncturing the skin to pool out blood thru vacuums; utilizing gravity-enhanced micro-channels to boost fluid collection in addition to capillary forces. In some embodiments, the device according to the invention will utilize a slight vacuum to pull blood from a capillary bed by capillary action instead of relying on a combination of capillary action and use of gravity as performed other blood collection systems such as the Hemolink system.

In other embodiments, a device according to the invention will use one or more non-mechanical pumps to collect and move blood, plasma or other fluids through the device. A non-mechanical pump functions using electro-hydrodynamic, electro-osmotic, or electrochemical forces, or by ultrasonic flow generation. Non-mechanical pumps that are chemically powered may have nano-motors fixed to surfaces to facilitate a chemical reaction powering the pump. In some embodiments of the invention, at least 2 or 3 microneedles are motivated by micro-pumps to apply an auto-pressure, thus avoiding external actuation. These embodiments of the invention avoid the need for mechanical systems containing moving parts such as actuation and valve membranes or flaps. The Hemolink system contains a mechanical actuator; upon depression this actuator applies mechanical pressure to deploy four lancets to pierce the skin and cause blood to be taken up. A driving force for a mechanical system can be generated using piezoelectric, electrostatic, thermo-pneumatic, pneumatic or magnetic effects. In other embodiments of the device according to the invention a combination of non-mechanical and mechanical pumps or drivers may be used.

Most embodiments of the invention will lack a detachable reservoir for holding collected blood, such as a reservoir holding about 150 μL of blood, which is mailed to an off-site laboratory, as used in the Hemolink system. However, unlike other blood collection systems, most embodiments of the invention will have a detection chamber containing antibodies producing a colorimetric signal when bound by hCG in blood plasma. However, in some embodiments, blood that is not processed through the system may be routed or placed in a separate container for off-site confirmation or processing.

Unlike other blood collection systems, the device of the invention has a microfluidic network configured to promote the flow of fluids from the collection site to the target destination, such as an outflow channel that extends into the detection area of the device of the invention.

Some conventional blood collection systems have microfluidic outflow channels that act as one-way flow values by retaining fluids and are configured to prevent backflow of fluid out of a tube or device, for example, Hemolink is configured to regulate the flow of fluids based on its orientation. In contrast, many embodiments of the invention use multiple valve pump systems to prevent the reverse flow of fluids and do not depend on the orientation of an internal microfluidic network.

Many embodiments of the device of the invention have both valves and channels that further extend the functionality of the microfluidic chip. Collected fluid can be transferred through channels such as open microfluidic channels in which capillary force dominates over gravity but they may also utilize a combination of capillary and gravity forces.

Some conventional blood systems are configured to include detachable tubes containing chemical additives such as EDTA, heparin, serum or a plasma separation gel. In contrast, many embodiments of the invention do not require such tubes or other detachable containers and the device will contain additives in or on a micro-filter or in or on other portions of the microfluidic chip.

In some embodiments, the device according to the invention will utilize a topical anesthetic (such as benzocaine, butamben, dibucaine, lidocaine, oxybuprocaine, pramoxine, proparacaine, proxymetacaine, or tetracaine), antibiotic, or disinfectant in the blood collection element to facilitate safe and painless removal of blood.

The terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

Links are disabled by insertion of a space or underlined space before “www” and may be reactivated by removal of the space.

As used herein, the singular forms “a”, “an” and “the” are intended to include the plural forms as well, unless the context clearly indicates otherwise.

As used herein, the term “and/or” includes any and all combinations of one or more of the associated listed items and may be abbreviated as “/”.

The terms “comprises” and/or “comprising,” when used in this specification, specify the presence of stated features, steps, operations, elements, and/or components, but do not preclude the presence or addition of one or more other features, steps, operations, elements, components, and/or groups thereof.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10%/a of the stated value (or range of values), +/−15% of the stated value (or range of values), +/−20% of the stated value (or range of values), etc. Any numerical range recited herein is intended to include all sub-ranges subsumed therein.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Disclosure of values and ranges of values for specific parameters (such as temperatures, molecular weights, weight percentages, etc.) are not exclusive of other values and ranges of values useful herein. Two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it is also envisioned that Parameter X may have other ranges of values including 1-9, 2-9, 3-8, 1-8, 1-3, 1-2, 2-10, 2.5-7.8, 2-8, 2-3, 3-10, and 3-9, as mere examples.

Although the open-ended term “comprising,” as a synonym of terms such as including, containing, or having, is use herein to describe and claim the present invention, the invention, or embodiments thereof, may alternatively be described using more limiting terms such as “consisting of” or “consisting essentially of” the recited ingredients. Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, define, in part, the scope of the foregoing claim terminology such that no inventive subject matter is dedicated to the public.

1. A device comprising a microfluidic chip comprising:
a chamber that when applied to a dermal capillary bed can be placed under a vacuum sufficient to remove whole blood from the capillary bed,
an inlet that receives non-venous blood from the chamber,
a filtration system for separating blood cells from plasma, wherein the filtration system comprises (i) a series of filters that remove blood cells, platelets or other solid components of blood ranging in diameter from 12 to 30 μm, 5 to <12 μm, and 0.1 to <5, or (ii) a cross-flow filtration system that separates plasma from whole blood;
a detecting region comprising antibodies that bind to a beta unit of human chorionic gonadotropin hormone; and
wherein the inlet and filtration system are connected so that blood flows from the inlet into the filtration system; and
wherein the filtration system and detecting region are connected so that plasma flows from the filtration system to the detecting region.

2. The device of claim 1, wherein the chamber is connected to a vacuum source outside of the microfluidic chip.

3. The device of claim 1 that comprises a series of micropumps that can drive blood or plasma when present in the chip through the filtration system and to the detecting region.

4. The device of claim 1 that comprises one or more structures that draw blood or plasma when present in the chip through the filtration system and to the detecting region.

5. The device of claim 1 that is connected to an external element that drives movement of blood or plasma when present in the chip through the filtration system by positive pressure applied upstream from the filtration system or by negative pressure provided downstream of the detecting region.

6. The device of claim 1 that comprises (i) the series of filters that remove blood cells, platelets or other solid components of blood ranging in diameter from 12 to 30 μm, 5 to <12 μm, and 0.1 to <5 μm.

7. The device of claim 1 that comprises (ii) the cross-flow filtration system that separates plasma from whole blood.

8. The device of claim 1 that comprises (ii) the cross-flow filtration system that separates plasma from whole blood and diaelectrophoretic electrodes that apply a charge to blood cells.

9. The device of claim 1, wherein the filtration system further comprises one or more outlets or reservoirs for removing blood cells, platelets, or other solid components of blood.

10. The device of claim 1, wherein flow of plasma from the cross-flow filtration system to the detecting region is driven by capillary forces.

11. The device of claim 1, wherein the detecting region emits a detectable signal when bound by the beta unit of human chorionic gonadotropin.

12. The device of claim 1, wherein the detecting region comprises an indicator that changes color when the detecting region is bound by the beta unit of human chorionic gonadotropin.

13. The device of claim 1, wherein the inlet, filtration system, or detecting region comprises at least one anti-coagulant.

14. The device of claim 1 that is configured to fit into a signal processing, display, data storage, or transmission device.

15. A system comprising the device of claim 1 and a vacuum source.

16. The system of claim 15 that further comprises an optical, radiation or other detector of a signal produced by binding of hCG in plasma to the antibodies in the detecting region.

17. The system of claim 15 that comprises an optical detector that is operatively connected to a screen that visually displays a result or that comprises an audio element that speaks or otherwise produces an auditory signal of a result, wherein said result is produced by binding of hCG in plasma to the antibodies in the detecting region.

18. The system of claim 15 that further comprises a signal processing, display, data storage, or transmission element.

19. The system of claim 15 that is in the form of a bracelet, watch, arm band, mobile phone, computer, or other wearable or handheld item.

20. A method for detecting human chorionic gonadotropin hormone in blood comprising contacting a blood sample with the device of claim 1 and detecting a signal generated when human chorionic gonadotropin binds to the detection region of said device.
